(19) United States
(12) Patent Application Publication
(10) Pub. No.: US 2024/0157024 A1
(43) Pub. Date: May 16, 2024

(54) CERAMIC SCAFFOLD

(71) Applicant: University of Southern California, Los Angeles, CA (US)

(72) Inventors: Xiangjia Li, Los Angeles, CA (US); Yong Chen, Los Angeles, CA (US); Yang Chai, Los Angeles, CA (US)

(21) Appl. No.: 17/755,462

(22) PCT Filed: Oct. 30, 2020
(86) PCT No.: PCT/US2020/058322

§ 371 Date: Apr. 29, 2022

Related U.S. Application Data
(60) Provisional application No. 62/929,630, filed on Nov. 1, 2019.

(51) Int. Cl. A61L 27/12; A61L 27/22; A61L 27/34

(52) U.S. Cl. CPC A61L 27/12; A61L 27/222; A61L 27/34; A61L 2430/02

(57) ABSTRACT

This disclosure generally relates to a ceramic scaffold. This disclosure particularly relates to a ceramic scaffold useful for bone regenerations. This disclosure also relates to a ceramic scaffold comprising hydroxyapatite (HA), tricalcium phosphate (TCP), or a mixture thereof. This disclosure also relates to a ceramic scaffold with high mechanical strength and flexibility. This disclosure further relates to a ceramic scaffold manufactured through a three-dimensional (3D) printing process, methods of manufacturing a ceramic scaffold and methods of replacing bone in a subject using the ceramic scaffold.

### CROSS-REFERENCE TO RELATED APPLICATION

This application claims the benefit of U.S. provisional patent application 62/929,630, entitled “Ceramic Scaffold,” filed Nov. 1, 2019, attorney docket number AMISC.012PR. The entire content of this application is incorporated herein by reference.

### BACKGROUND

### Technical Field

This disclosure generally relates to a ceramic scaffold. This DISCLOSURE particularly relates to a ceramic scaffold useful for bone regenerations. This disclosure also relates to a ceramic scaffold comprising hydroxyapatite (HA), tricalcium phosphate (TCP), or a mixture thereof. This disclosure also relates to a ceramic scaffold with high mechanical strength and flexibility. This disclosure further relates to a ceramic scaffold manufactured through a three-dimensional (3D) printing process.

### Description of Related Art

Over millions of patients require surgical reconstruction of bone defects, which are caused by trauma, tumor, or infections, etc., worldwide each year [1-3]. Common treatment methods, including bone autografts [4], allograft [4], and substitute material-based implant [5], still have certain limitations to treat the bone injury [6]. For example, the autografts can be applied only to the bone defects with the small area due to quantities constraints, and it also may cause a high risk in procurement morbidity [7]. With the development of tissue engineering, a new bone defect treatment method, constructing the microenvironment of bone tissue by integrating extracellular matrix, cells, growth factor, etc., was put forward to regrowth the new bone for the healing of bone defects [8]. To reproduce the natural living matter, the 3D scaffold was further designed and fabricated using biocompatible and biodegradable materials [9]. Benefit from the improvement of the fabrication capability of additive manufacturing (AM), the 3D scaffold with complex inner microstructures can be achieved [10, 11]. Along with the progress in AM technologies, an increasing number of materials, such as bio-ceramic, polymer, hydrogel, and nanocomposite, can be reproduced to mimic natural bone tissue [12]. Among these biomaterials, bioceramic-like hydroxyapatite (HA) and tricalcium phosphate (TCP), exhibits promising performance in cell attachment and proliferation [13], osteoconduction [14], osseointegration and osteoinduction [16]. However, HA/TCP scaffolds, which were fabricated by traditional methods [8-9], were too fragile to conduct further manipulations. The poor mechanical performance limits the scope of the bioceramic scaffold in bone tissue regeneration. As we know, the compressive strength of natural trabecular and cortical bone reach 100-130 MPa and 130-190 MPa respectively [17], and some researches show that the 3D scaffold with similar bone mechanical characteristics can promote bone regeneration [17-20]. Various research studies have been conducted to improve the mechanical performance of HA/TCP scaffold, such as, increasing the sintering temperature [21], or combining with other composition [22]. However, even though the mechanical strength was increased, the degradation of the scaffold was slowed down, which may cause more serious risks in infection and cancelation [23]. Ideally, the 3D printed scaffold should gradually degrade during the regrowth of new bone, and meanwhile, the residual 3D scaffold still can maintain the shape with certain mechanical strength [24, 25]. Overall, most of the scaffolds, which are fabricated by using current manufacturing methods, cannot meet the tradeoff between mechanical performance and degradation.

### SUMMARY

This disclosure generally relates to a ceramic scaffold. This disclosure particularly relates to a ceramic scaffold useful for bone regenerations. This disclosure also relates to a ceramic scaffold comprising hydroxyapatite (HA), tricalcium phosphate (TCP), or a mixture thereof. This disclosure also relates to a ceramic scaffold with high mechanical strength and flexibility. This disclosure further relates to a ceramic scaffold manufactured through a three-dimensional (3D) printing process.

In this disclosure, a ceramic scaffold may include a framework and a coating. The framework may include hydroxyapatite (HA), tricalcium phosphate (TCP), or a mixture thereof. The coating may include a polymer (“coating polymer”).

In this disclosure, the framework may have at least one surface. The coating may be formed on the at least one surface of the framework. The coating may at least partially cover the at least one surface of the framework. Or, the coating may substantially cover the at least one surface of the framework.

In this disclosure, the coating polymer may include a polymer, which may be formed by using a surgical glue, a gelatinous protein mixture, poly(ethylene glycol) dimethacrylate (PEGDMA), gelatin methacrylate (GelMA), gelatin, or a mixture thereof. Or, the coating polymer may include a polymer formed by using a surgical glue. In some embodiments, the surgical glue polymer is 2-octyl cyanoacrylate (Dermabond). Or, the coating polymer may include a gelatin. Or, the coating polymer may include a polymer formed by using a cyanoacrylate glue, a fibrin sealant, a collagen-based compound, a glutaraldehyde glue, a hydrogel, or a mixture thereof. Or, the coating polymer may include a polymer formed by using n-butyl cyanoacrylate, 2-octyl cyanoacrylate, or a mixture thereof.

In this disclosure, mechanical strength of the ceramic scaffold may be in a range of 15 N to 33 N when measured as a maximum load of the stress vs. strain curve. For example, the mechanical strength may be 15 N, 16 N, 17 N, 18 N, 19 N, 20 N, 21 N, 22 N, 23 N, 24 N, 25 N, 26 N, 27 N, 28 N, 29 N, 30 N, 31 N, 32 N or 33 N or any range defined by these values. The flexural strength of the ceramic scaffold may be in a range of 10 MPa to 50 MPa, including values of 10 mPa, 11 MPa, 12 MPa, 13 MPa, 14 MPa, 15 MPa, 16 MPa, 17 MPa, 18 MPa, 19 MPa, 20 MPa, 21 MPa, 22 MPa, 23 MPa, 24 MPa, 25 MPa, 26 MPa, 27 MPa, 28 MPa, 29 MPa, 30 MPa, 31 MPa, 32 MPa, 33 MPa, 34 MPa, 35 MPa, 36 MPa, 37 MPa, 38 MPa, 39 MPa, 40 MPa, 41 MPa, 42 MPa, 43 MPa, 44 MPa, 45 MPa, 46 MPa, 47 MPa, 48 MPa, 49 MPa or 50 MPa or any range defined by these values; wherein the flexural strength is measured by a standard three-point bending test. Or, mechanical strength of the ceramic scaffold may be at least 5 times higher than that of the framework. Or, mechanical strength of the ceramic scaffold may be at least 10 times higher than that of the framework. Or, mechanical strength of the ceramic scaffold may be 10 times to 20 times higher than that of the framework.

In this disclosure, thickness of the coating polymer may be in a range of 1 micrometer to 1,000 micrometers. Or, thickness of the coating polymer may be in a range of 10 micrometers to 500 micrometers.

This disclosure also relates to a method of manufacturing a ceramic scaffold. This method may include preparing a slurry including hydroxyapatite (HA), tricalcium phosphate (TCP), or a mixture thereof; and a UV polymerizable monomer formulation; using a three dimensional (3D) printing process and the slurry to prepare a green body; debinding and sintering the green body to remove the polymer formed by polymerization of the UV polymerizable monomer formulation to prepare a sintered porous body, wherein the sintered porous body forms the framework; coating the sintered porous body with a polymer coating solution; polymerizing the polymer coating solution to form a coating including a polymer (“coating polymer”); and thereby obtaining the ceramic scaffold.

In this disclosure, the three-dimensional (3D) printing process may be a mask image projection-based slurry printing (MIP-SP) process.

In this disclosure, the ceramic scaffold may be sintered at a temperature in a range of 1,000 centigrade to 1,500 centigrade. Or, wherein the green body may be sintered at a temperature in the range of 1,050 centigrade to 1,250 centigrade.

In this disclosure, the coating of the sintered porous body may be carried out by a process that may include a surface spraying process, a brush spraying process, a vacuum merging process, or a combination thereof.

Any combination of above ceramics scaffolds and/or methods of preparation of these ceramic scaffolds is within the scope of this disclosure.

Some aspects relate to a ceramic scaffold, including:
a framework including hydroxyapatite (HA), tricalcium phosphate TCP), or a mixture thereof; and
a coating including a coating polymer;
wherein the framework has at least one surface, wherein the coating is formed on the at least one surface of the framework, and wherein the coating at least partially covers the at least one surface of the framework.

In some examples, the coating polymer includes a polymer formed by using a surgical glue, a gelatinous protein mixture, poly(ethylene glycol) dimethacrylate (PEGDMA), gelatin methacrylate (GelMA), a gelatin, or a mixture thereof.

In some examples, the coating polymer includes a surgical glue, a gelation, or a mixture thereof.

In some examples, the coating polymer includes a polymer of an n-butyl cyanoacrylate monomer, a 2-octyl cyanoacrylate monomer, or a mixture thereof.

In some examples, the coating is coated onto the surface of the scaffold at a thickness of from 5 μm to 1 mm.

In some examples, a mechanical strength of the ceramic scaffold is in a range of 15 N to 33 N when measured as a maximum load of the stress vs. strain curve.

In some examples, a flexural strength of the ceramic scaffold is in the range of 10 MPa to 50 MPa; wherein the flexural strength is measured by a standard three-point bending test.

In some examples, the mechanical strength of the ceramic scaffold is 10 times to 20 times higher than that of the framework.

In some examples, the coating polymer includes a polymer formed by using a cyanoacrylate glue, a fibrin sealant, a collagen-based compound, a glutaraldehyde glue, a hydrogel, or a mixture thereof.

In some examples, the coating polymer includes an acrylate polymer.

In some examples, the coating polymer includes a cyanoacrylate polymer.

In some examples, the coating polymer includes a surgical glue.

In some examples, the coating polymer includes a gelatin.

In some examples, the coating polymer includes a polymer of an n-butyl cyanoacrylate monomer, a 2-octyl cyanoacrylate monomer, or a mixture thereof.

In some examples, the coating is coated onto the surface of the scaffold at a thickness of from 5 μm to 1 mm.

In some examples, the scaffold includes stacked layers of the hydroxyapatite (HA), tricalcium phosphate (TCP), or a mixture thereof, each layer having a thickness of from 10 μm to 200 μm.

In some examples, the mechanical strength of the ceramic scaffold is in a range of 15 N to 33 N when measured as a maximum load of the stress vs. strain curve.

In some examples, the flexural strength of the ceramic scaffold is in the range of 10 MPa to 50 MPa; wherein the flexural strength is measured by a standard three-point bending test.

In some examples, a mechanical strength of the ceramic scaffold is at least 5 times higher than that of the framework.

In some examples, the mechanical strength of the ceramic scaffold is at least 10 times higher than that of the framework.

In some examples, the mechanical strength of the ceramic scaffold is 10 times to 20 times higher than that of the framework.

In some examples, a thickness of the coating polymer is in a range of 1 micrometer to 1,000 micrometers.

In some examples, a thickness of the coating polymer is in a range of 10 micrometers to 500 micrometers.

In some examples, the coating polymer includes a polymer formed by using a surgical glue, a gelatinous protein mixture, polyethylene glycol) dimethacrylate (PEGDMA), gelatin methacrylate (GelMA), gelatin, or a mixture thereof; wherein mechanical strength of the ceramic scaffold is in a range of 15 N to 33 N when measured as a maximum load of the stress vs. strain curve, and/or flexural strength of the ceramic scaffold is in the range of 10 MPa to 50 MPa; and wherein the flexural strength is measured by a standard three-point bending test.

In some examples, the coating polymer includes a polymer formed by using a surgical glue, a gelatinous protein mixture, polyethylene glycol) dimethacrylate (PEGDMA), gelatin methacrylate (GelMA), gelatin, or a mixture thereof; wherein mechanical strength of the ceramic scaffold is at least 5 times higher than that of the framework, or at least 10 times higher than that of the framework, or 10 times to 20 times higher than that of the framework; and wherein the mechanical strength of the ceramic scaffold is a maximum load of the stress vs. strain curve.

In some examples, the coating polymer includes a polymer formed by using n-butyl cyanoacrylate, 2-octyl cyanoacrylate, or a mixture thereof; wherein mechanical strength of the ceramic scaffold is in a range of 15 N to 33 N when measured as a maximum load of the stress vs. strain curve, and/or flexural strength of the ceramic scaffold is in the range of 10 MPa to 50 MPa; and wherein the flexural strength is measured by a standard three-point bending test.

In some examples, the coating polymer induces a polymer formed by using n-butyl cyanoacrylate, 2-octyl cyanoacrylate, or a mixture thereof; wherein mechanical strength of the ceramic scaffold is at least 5 times higher than that of the framework, or at least 10 times higher than that of the framework, or 10 times to 20 times higher than that of the framework; and wherein the mechanical strength of the ceramic scaffold is a maximum load of the stress vs. strain curve.

In some examples, the coating substantially covers the at least one surface of the framework.

In some examples, the coating polymer includes a polymer formed by using a surgical glue, a gelatinous protein mixture, polyethylene glycol) dimethacrylate (PEGDMA), gelatin methacrylate (GelMA), a gelatin, or a mixture thereof.

In some examples, the coating polymer includes a polymer formed by using a surgical glue.

In some examples, the coating polymer includes a polymer formed by using a gelatin.

In some examples, the coating polymer includes a polymer formed by using a cyanoacrylate glue, a fibrin sealant, a collagen-based compound, a glutaraldehyde glue, a hydrogel, or a mixture thereof.

In some examples, the coating polymer includes a polymer formed by using n-butyl cyanoacrylate, 2-octyl cyanoacrylate, or a mixture thereof.

In some examples, a mechanical strength of the ceramic scaffold is in a range of 15 N to 33 N when measured as a maximum load of the stress vs. strain curve.

In some examples, a flexural strength of the ceramic scaffold is in the range of 10 MPa to 50 MPa; wherein the flexural strength is measured by a standard three-point bending test.

In some examples, a mechanical strength of the ceramic scaffold is at least 5 times higher than that of the framework.

In some examples, the mechanical strength of the ceramic scaffold is at least 10 times higher than that of the framework.

In some examples, the mechanical strength of the ceramic scaffold is 10 times to 20 times higher than that of the framework.

In some examples, a thickness of the coating polymer is in a range of 1 micrometer to 1,000 micrometers.

In some examples, a thickness of the coating polymer is in a range of 10 micrometers to 500 micrometers.

In some examples, the coating polymer includes a polymer formed by using a surgical glue, a gelatinous protein mixture, poly(ethylene glycol) dimethacrylate (PEGDMA), gelatin methacrylate (GelMA), gelatin, or a mixture thereof; wherein mechanical strength of the ceramic scaffold is in a range of 15 N to 33 N when measured as a maximum load of the stress vs. strain curve, and/or flexural strength of the ceramic scaffold is in the range of 10 MPa to 50 MPa; and wherein the flexural strength is measured by a standard three-point bending test.

In some examples, the coating polymer includes a polymer formed by using a surgical glue, a gelatinous protein mixture, poly(ethylene glycol) dimethacrylate (PEGDMA), gelatin methacrylate (GelMA), gelatin, or a mixture thereof; wherein mechanical strength of the ceramic scaffold is at least 5 times higher than that of the framework, or at least 10 times higher than that of the framework, or 10 times to 20 times higher than that of the framework; and wherein the mechanical strength of the ceramic scaffold is a maximum load of the stress vs. strain curve.

In some examples, the coating polymer includes a polymer formed by using n-butylcyanoacrylate, 2-octyl cyanoacrylate, or a mixture thereof; wherein mechanical strength of the ceramic scaffold is in a range of 15 N to 33 N when measured as a maximum load of the stress vs. strain curve, and/or flexural strength of the ceramic scaffold is in the range of 10 MPa to 50 MPa; and wherein the flexural strength is measured by a standard three-point bending test.

In some examples, the coating polymer includes a polymer formed by using n-butyl cyanoacrylate, 2-octyl cyanoacrylate, or a mixture thereof; wherein mechanical strength of the ceramic scaffold is at least 5 times higher than that of the framework, or at least 10 times higher than that of the framework, or 10 times to 20 times higher than that of the framework; and wherein the mechanical strength of the ceramic scaffold is a maximum load of the stress vs. strain curve.

In some examples, the coating substantially covers the at least one surface of the framework

Some aspects relate to a method of manufacturing a ceramic scaffold, including:
preparing a slurry including hydroxyapatite (HA), tricalcium phosphate (TCP), or a mixture thereof; and a UV polymerizable monomer formulation;
using a three-dimensional (3D) printing process and the slurry to prepare a green body;
debinding and sintering the green body to remove the polymer formed by polymerization of the UV polymerizable monomer formulation to prepare a sintered porous body, wherein the sintered porous body forms the framework;
coating the sintered porous body with a polymer coating solution;
polymerizing the polymer coating solution to form a coating including a coating polymer; and thereby
obtaining the ceramic scaffold.

In some examples, the three-dimensional (3D) printing process is a mask image projection-based slurry printing (MIP-SP) process.

In some examples, the green body is sintered at a temperature in a range of 1,000 centigrade to 1,500 centigrade.

In some examples, the green body is sintered at a temperature in the range of 1,050 centigrade to 1,250 centigrade.

In some examples, the coating of the sintered porous body is carried out by a process including a surface spraying process, a brush spraying process, a vacuum merging process, or a combination thereof.

In some examples, the ceramic scaffold obtained is a ceramic scaffold of any of claims 1 to 45.

In some examples, the coating polymer includes a polymer formed by using a surgical glue, a gelatinous protein mixture, poly(ethylene glycol) dimethacrylate (PEGDMA), gelatin methacrylate (GelMA), gelatin, or a mixture thereof.

In some examples, the coating polymer includes a polymer formed by using a surgical glue.

In some examples, the coating polymer includes a polymer formed by using a gelatin.

In some examples, the coating polymer includes a polymer formed by using a cyanoacrylate glue, a fibrin sealant, a collagen-based compound, a glutaraldehyde glue, a hydrogel, or a mixture thereof.

In some examples, the coating polymer includes a polymer formed by using n-butyl cyanoacrylate, 2-octyl cyanoacrylate, or a mixture thereof.

In some examples, the three-dimensional (3D) printing process is a mask image projection-based slurry printing (MIP-SP) process.

In some examples, the green body is sintered at a temperature in a range of 1,000 centigrade to 1,500 centigrade.

In some examples, the green body is sintered at a temperature in the range of 1,050 centigrade to 1,250 centigrade.

In some examples, the coating of the sintered porous body is carried out by a process including a surface spraying process, a brush spraying process, a vacuum merging process, or a combination thereof.

In some examples, the ceramic scaffold obtained is a ceramic scaffold of any of claims 1 to 45.

In some examples, the coating polymer includes a polymer formed by using a surgical glue, a gelatinous protein mixture, polyethylene glycol) dimethacrylate (PEGDMA), gelatin methacrylate (GelMA), gelatin, or a mixture thereof.

In some examples, the coating polymer includes a polymer formed by using a surgical glue.

In some examples, the coating polymer includes a polymer formed by using a gelatin.

In some examples, the coating polymer includes a polymer formed by using a cyanoacrylate glue, a fibrin sealant, a collagen-based compound, a glutaraldehyde glue, a hydrogel, or a mixture thereof.

In some examples, the coating polymer includes a polymer formed by using n-butyl cyanoacrylate, 2-octyl cyanoacrylate, or a mixture thereof.

In some examples, the debinding is done under a vacuum at a pressure of 0.01-0.5 MPa.

In some examples, the coating is applied to the scaffold under a vacuum.

In some examples, the coating is applied to the scaffold by surface spraying.

Some aspects relate to a method of replacing bone in a subject, including:
identifying the subject as missing a portion of bone or removing a portion of bone from the subject, and
placing a ceramic scaffold according to any one of claims 1-8 in place of the missing or removed portion of bone, wherein the ceramic scaffold allows for regeneration of bone in and around the ceramic scaffold.

Some aspects relate to a method of replacing bone in a subject, including:
identifying the subject as missing a portion of bone or removing a portion of bone from the subject, and
placing a ceramic scaffold according to any one of claims 1-45 in place of the missing or removed portion of bone, wherein the ceramic scaffold allows for regeneration of bone in and around the ceramic scaffold.

Any combination of the above aspects and examples is within the scope of what we claim.

These, as well as other components, steps, features, objects, benefits, and advantages, will now become clear from a review of the following detailed description of illustrative examples, the accompanying drawings, and the claims.

### BRIEF DESCRIPTION OF DRAWINGS

The drawings are of illustrative examples. They do not illustrate all examples. Other examples may be used in addition or instead. Details that may be apparent or unnecessary may be omitted to save space or for more effective illustration. Some examples may be practiced with additional components or steps and/or without all of the components or steps that are illustrated. When the same numeral appears in different drawings, it refers to the same or like components or steps.

The patent or application file contains at least one drawing executed in color. Copies of this patent or patent application publication with color drawing(s) will be provided by the Office upon request and payment of the necessary fee.

FIG. 1. The fabrication procedures of HA/TCP scaffold with reinforcement for bone defects. (a) Bone fracture and critical defect; (b) the ingredient of HA/TCP slurry; (c) the schematic diagram of MIP-SP process for the fabrication of HA/TCP green part; (d) the green part of 3D printed HA/TCP scaffold; (e) the brown part of HA/TCP scaffold after debinding process; (f) the pure HA/TCP scaffold after sintering process; (g) the schematic diagram of vacuum coating processing; (h) HA/TCP scaffold after coating process; and (i) trimming the HA/TCP scaffold based on the shape of bone fracture and critical defect.

FIG. 2. The effect of coating material on the mechanical performance of the HA/TCP microcell structure. (a) The schematic diagram of second stage coating process by using vacuum merging; (b, c) the full view and scanning electron microscopy (SEM) image of the HA/TCP microcell without coating and with different coating materials; and (d) the max load comparison of HA/TCP microcell structures with and without coating.

FIG. 3. Mechanical reinforcement of HA/TCP scaffold by post-coating process. (a) Schematic diagram of post-coating process; (b-c) full view and SEM (scanning electron microscopy) images of HA/TCP scaffolds before and after coating process; (d) the section view of HA/TCP microcell structures with different coating process and coating parameters; (e) the maximum load of HA/TCP microcell structures can withstand after the reinforcement of different coating methods; (f) the fracture toughness of HA/TCP printed parts processed with different coating methods; and (g) the shape changing of 3D printed HA/TCP parts after the sintering and coating process.

FIG. 4. The compression properties of 3D printed HA/TCP parts before and after coating process. (a) The coating material was filled inside the pores between HA/TCP particles after coating process; (b-c) simulation results of stress distribution of 3D printed HA/TCP parts before and after coating process by using COMSOL Multiphysics, respectively; (d) crack deflection only along the HA/TCP particles without coating material; (e) simulation results of stress distribution of 3D printed HA/TCP parts sintered at different temperatures after coating process by using COMSOL Multiphysics, respectively; (f) the compression properties of 3D printed HA/TCP microcell structures sintered at 1250 ° C. with and without the coating of surgical glue; and (g) the compression properties of 3D printed HA/TCP parts sintered at different temperatures with and without the coating of surgical glue.

FIG. 5. The comparisons of flexural strength, fracture toughness, and tensile strength of 3D printed HA/TCP parts before and after the coating process. (a) The three point bending testing of HA/TCP printed parts; (b-d) the load-displacement, fracture toughness and flexural strength of 3D printed HA/TCP parts with and without coating material; (e) simulations of stress distribution by COMSOL Multiphysics for the 3D printed HA/TCP parts with and without coating material; SEM images showing the fracture surfaces in the pure 3D printed HA/TCP part (f) and the 3D printed HA/TCP part with surgical glue coating (g); (h) the tensile testing of 3D printed HA/TCP parts; (i, j) the stain-stress and Young's modulus of 3D printed HA/TCP part sintered at different temperature with and without coating material; and (k) the simulation appearance of 3D printed HA/TCP parts sintered at low and high temperatures respectively by using COMSOL Multiphysics.

FIG. 6. The design and fabrication of 3D trimmable HA/TCP plate. (a) The CAD model of 3D trimmable HA/TCP plate; the images of 3D printed HA/TCP plate (b) before and after (c) debinding and sintering; (d) with surgical glue coating; (e) the trimming process of HA/TCP plate; (f) the cut HA/TCP plate for the cranial facial defect; (g) the SEM image of HA/TCP plate; and (h) the metal screws were mounted on the trimmable HA/TCP plate for the fixture.

FIG. 7. The cranial facial bone reconstruction by 3D printed HA/TCP scaffolds with coating reinforcement. (a) The cranial facial bone defect animal model; (b) CAD model of the HA/TCP scaffold for the critical cranial facial bone defect; the images of 3D printed HA/TCP scaffold before (c) and after (d) the debinding and sintering; (e) with surgical glue coating; (f) compression simulation of HA/TCP scaffold using COMSOL Multiphysics; and compression test of 3D printed HA/TCP scaffold (g) without and (h) with coating; and (i) the force and displacement of HA/TCP scaffold with and without coating in the compression test.

FIG. 8. The design and fabrication of HA/TCP scaffolds for long bone defect reconstruction. (a) The digital model of critical mouse femur defect animal model; (b) CAD model of scaffold for long bone defect; the images of 3D printed HA/TCP scaffold before (c) and after (d) debinding and sintering; (e) the compression simulation of the HA/TCP scaffold using COMSOL Multiphysics; (f) the image of 3D printed HA/TCP scaffold with surgical glue coating; and (g) the comparison of maximum loads that the HA/TCP scaffolds can stand with different fabrication parameters.

### DETAILED DESCRIPTION OF ILLUSTRATIVE EXAMPLES

Illustrative examples are now described. Other examples may be used in addition or instead. Details that may be apparent or unnecessary may be omitted to save space or for a more effective presentation. Some examples may be practiced with additional components or steps and/or without all of the components or steps that are described.

This disclosure generally relates to a ceramic scaffold. This disclosure particularly relates to a ceramic scaffold useful for bone regenerations. This disclosure also relates to a ceramic scaffold comprising hydroxyapatite (HA), tricalcium phosphate (TCP), or a mixture thereof. This disclosure also relates to a ceramic scaffold with high mechanical strength and flexibility. This disclosure further relates to a ceramic scaffold manufactured through a three-dimensional (3D) printing process.

This disclosure generally relates to a ceramic scaffold. This disclosure particularly relates to a ceramic scaffold useful for bone regenerations. This disclosure also relates to a ceramic scaffold comprising hydroxyapatite (HA), tricalcium phosphate (TCP), or a mixture thereof. This disclosure also relates to a ceramic scaffold with high mechanical strength and flexibility. This disclosure further relates to a ceramic scaffold manufactured through a three-dimensional (3D) printing process.

In this disclosure, a ceramic scaffold may include a framework and a coating. The framework may include hydroxyapatite (HA), tricalcium phosphate (TCP), or a mixture thereof. The coating may include a polymer (“coating polymer”).

In this disclosure, the framework may have at least one surface. The coating may be formed on the at least one surface of the framework. The coating may at least partially cover the at least one surface of the framework. Or, the coating may substantially cover the at least one surface of the framework.

In this disclosure, the coating polymer may include a polymer, which may be formed by using a surgical glue, a gelatinous protein mixture, poly(ethylene glycol) dimethacrylate (PEGDMA), gelatin methacrylate (GelMA), gelatin, or a mixture thereof. Or, the coating polymer may include a polymer formed by using a surgical glue. Or, the coating polymer may include a gelatin. Or, the coating polymer may include a polymer formed by using a cyanoacrylate glue, a fibrin sealant, a collagen-based compound, a glutaraldehyde glue, a hydrogel, or a mixture thereof. Or, the coating polymer may include a polymer formed by using n-butyl cyanoacrylate, 2-octyl cyanoacrylate, or a mixture thereof.

In this disclosure, mechanical strength of the ceramic scaffold may be in a range of 15 N to 33 N when measured as a maximum load of the stress vs. strain curve. And/or flexural strength of the ceramic scaffold may be in a range of 10 MPa to 50 MPa; wherein the flexural strength is measured by a standard three-point bending test. Or, mechanical strength of the ceramic scaffold may be at least 5 times higher than that of the framework. Or, mechanical strength of the ceramic scaffold may be at least 10 times higher than that of the framework. Or, mechanical strength of the ceramic scaffold may be 10 times to 20 times higher than that of the framework.

In this disclosure, thickness of the coating polymer may be in a range of 1 micrometer to 1,000 micrometers. Or, thickness of the coating polymer may be in a range of 10 micrometers to 500 micrometers.

This disclosure also relates to a method of manufacturing a ceramic scaffold. This method may comprise preparing a slurry comprising hydroxyapatite (HA), tricalcium phosphate (TCP), or a mixture thereof; and a UV polymerizable monomer formulation; using a three dimensional (3D) printing process and the slurry to prepare a green body; debinding and sintering the green body to remove the polymer formed by polymerization of the UV polymerizable monomer formulation to prepare a sintered porous body, wherein the sintered porous body forms the framework; coating the sintered porous body with a polymer coating solution; polymerizing the polymer coating solution to form a coating comprising a polymer (“coating polymer”); and thereby obtaining the ceramic scaffold.

In this disclosure, the three-dimensional (3D) printing process may be a mask image projection-based slurry printing (MIP-SP) process.

In this disclosure, the ceramic scaffold may be sintered at a temperature in a range of 1,000 centigrade to 1,500 centigrade. Or, wherein the green body may be sintered at a temperature in the range of 1,050 centigrade to 1,250 centigrade.

In this disclosure, the coating of the sintered porous body may be carried out by a process that may include a surface spraying process, a brush spraying process, a vacuum merging process, or a combination thereof.

Any combination of above ceramics scaffolds and/or methods of preparation of these ceramic scaffolds is within the scope of this disclosure.

The bone defect is one of the hardest-to-heal injuries that bother the medical profession and seriously affects patients' life quality for many years. Millions of surgical operations were conducted annularly by using traditional gold standard graft, where the significant limitation in the dimension and osteogenesis is still ubiquitous. The three-dimensional (3D) scaffold-based therapeutic approach provides a promising solution that the new bone can be regenerated with the help of osteoblast. Both the mechanical strength and degradation of scaffolds play crucial roles in bone tissue regeneration. However, there is still a great challenge in the development of scaffold for bone regeneration due to the tradeoff between mechanical performance and degradation. Herein, a new 3D printing integrated hybrid process was investigated to not only efficiently increase the mechanical performance hundreds of times and but also achieve the desired degradation speed of 3D printed scaffolds. The experiments were conducted to study the impact of material selection and process planning on the mechanical and degradation performances of the bioceramic scaffold. Specific 3D scaffold designed for long bone and cranial facial circle defects were designed and fabricated by using this proposed method. Based on our newly developed manufacturing method, a 3D trimmable plate was further invented to generate a universal and low-cost solution for bone tissue regeneration. Such an approach opens intriguing perspectives in the treatment of bone defects with the potential to be integrated into scalable manufacturing.

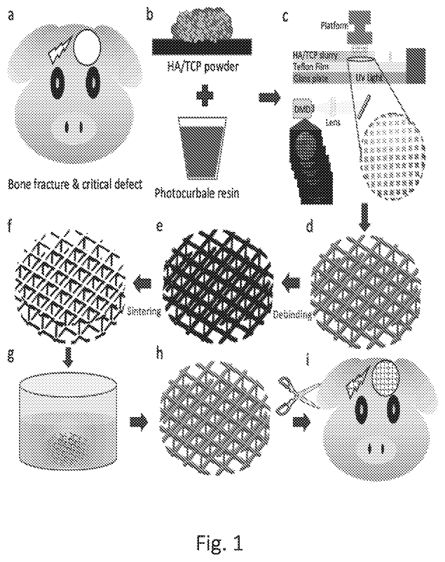

To solve these challenges, a 3D printing method with reinforcement coating was developed to manufacture bioceramic scaffold (FIG. 1). Firstly, green parts of HA/TCP scaffolds were fabricated using the mask image projection-based slurry printing (MIP-SP). In some examples, the scaffold is printed with successive layers, each layer set at a thickness of from 10 μm to 200 μm, for example, 10 μm, 20 μm, 30 μm, 40 μm, 50 μm, 60 μm, 70 μm, 80 μm, 90 μm, 100 μm, 120 μm, 140 μm, 160 μm, 180 μm or 200 μm. After that, HA/TCP scaffolds were processed by the debinding and sintering to remove the inner polymer and fuse together the HA/TCP particles, generating microscale porous structures inside the scaffold. Then the biocompatible material, whose degradation speed is faster than that of HA/TCP, was coated inside the HA/TCP scaffold, and the coating depth can be controlled by adjusted the values of different coating parameters. The mechanical performance of the HA/TCP scaffold was improved dramatically along with the biodegradable coating material penetrating the HA/TCP scaffold and filling inside the blank area between the HA/TCP particles. By integrating the coating process with the MIP-SP, it is possible to achieve higher mechanical strength with lower sintering temperature, enabling faster degradation of the HA/TCP scaffold. It opens a new means to achieve both high mechanical properties and fast degradation at the same time.

To address problems in the reinforcement of the HA/TCP scaffold, we firstly determined the roles of the coating process on the mechanical performance and degradation of the HA/TCP scaffold. Accordingly, we identified the reinforcement mechanism of the coating process in the HA/TCP scaffold fabrication. After that, the material selection and process parameters were investigated and optimized to modify the features of the HA/TCP scaffold. Two types of HA/TCP scaffolds were designed and fabricated for critical bone defects, including long bone and cranial facial bone, to demonstrate the feasibility of our 3D printing with the reinforcement approach.

The ceramic scaffolds disclosed herein may be used in any type of bone replacement, including but not limited to the following types of bone: spinal bones, including cervical vertebrae, thoracic vertebrae, lumbar vertebrae, sacrum, coccygeal vertebrae/cordal; chest (thorax), hyoid bone, sternum, ribs; skull including the bones of the middle ear, head bones including cranial hones, occipital bone, parietal bones, frontal bone, temporal bones, sphenoid bone, ethmoid bone, facial bones, nasal bones, maxillae (upper jaw), lacrimal bone, zygomatic bone (cheek bones), palatine bone, inferior nasal concha, vomer, mandible, middle ear, malleus, incus, stapes; arm bones including: humerus, pectoral girdle (shoulder), scapula, clavicles, ulna, radius; hand bones including carpals, scaphoid bone, lunate bone, triquetral bone, pisiform bone, trapezium, trapezoid bone, capitate bone, hamate bone, metacarpals, phalanges of the hand, proximal phalanges, intermediate phalanges, distal phalanges; pelvis (pelvic girdle), ilium, ischium, and pubis; leg bones including femur, patella or kneecap, tibia, fibula; foot bones including tarsus/tarsals, calcaneus or heel bone, talus, navicular bone, medial cuneiform bone, intermediate cuneiform bone, lateral cuneiform bone, cuboid bone, metatarsals, phalanges of the foot, proximal phalanges, intermediate phalanges, and distal phalanges.

Moreover, HA/TCP scaffolds have been designed for the treatment and study of bone defects, which open intriguing perspectives for the clinical trials. However, an effective large-scale design and manufacturing methods of HA/TCP scaffold for bone defects are still scarce. Benefit from the coating process, a 3D printed trimmable HA/TCP scaffold was designed and fabricated by using our developed method. It provides a new means to fill the gap between customization and mass production. Our proposed method addresses the current limitation of the HA/TCP scaffold with the feasibility of manual manipulation for different purposes. The biological tests including biocompatibility and cell attachment tests were conducted to present the greater manipulability and translational potential of our proposed method in the large-scale bone defect treatments.

Bioceramic HA/TCP shows the superiority and advantages in cell attachment and proliferation, osteoconduction, osseointegration, and osteoinduction comparing with other types of biomaterial. However, the poor mechanical performance of the HA/TCP scaffold has restricted its wide applications in bone tissue regeneration. In this work, a new 3D printing with reinforcement coating approach was put forward so that the mechanical performances of the 3D printed HA/TCP scaffold can be significantly improved without affecting the degradation of the HA/TCP scaffold. The impact of the reinforcement process on the mechanical performances and degradation of the scaffold was modulated by choosing the coating material and controlling the coating duration and procedures. Two types of the post coating process, including surface spraying and vacuum merging, were applied to achieve different levels of mechanical reinforcement. The mechanism of the coating reinforcement was identified and validated by both theoretical and experimental analysis. The HA/TCP scaffold with special coating shows remarkable mechanical property and degradation compared with the traditional approaches. The 3D HA/TCP scaffolds were designed and fabricated using our developed method for long bone and cranial facial critical bone defects to evaluate the degradation and mechanical performance. Moreover, the universal HA/TCP trimmable scaffold was built, providing a possibility of large-scale manufacturing of the HA/TCP scaffold for the general bone regeneration purpose. It opens intriguing perspectives for designing HA/TCP scaffold based on the reinforcement coating process to form greater manipulability and translational potential of HA/TCP scaffold that eliminating the bottleneck in the current applications in tissue engineering.

### EXAMPLE 1. MIP-SP of 3D HA/TCP SCAFFOLD

It is challenging to fabricate bioceramic scaffold with a controllable distribution of microscale structures by using the traditional manufacturing methods, such as freezing cast, foam replica, particle leaching, and injection molding, etc. Additive manufacturing technologies exhibit advantages in building customized 3D scaffolds from scratch using different kinds of biomaterial. In this work, green parts of 3D HA/TCP scaffolds were firstly fabricated using the self-developed MIP-SP process. In the 3D printing of the green part, the digital model of the 3D scaffold was sliced to get a set of two dimensional (2D) mask images with 75 μm layer thickness, which was decided by the cure depth of the HA/TCP slurry (FIG. 1, c). The generated mask image was used to indicate the digital micromirrors to project the light with a 2D pattern, and the grayscale of each pixel in the mask image was adjusted to further control the light intensity. The uniformed light beam penetrated the transparent glass disc from bottom and was focused on the top surface. After receiving enough energy from the exposure of a 2D patterned light beam, a layer of HA/TCP slurry selectively transferred from the liquid phase to solid phase due to the crosslink reaction of photocurable polymer, resulting in the HA/TCP particles sealing inside the solidified layer. After building one layer, the platform moved up, and a new layer of HA/TCP slurry was fed at the light projection area along with the rotation of transparent disc by using the blade-assisted material feeding system. The platform then moved down to form a uniform layer of slurry for the coming layer fabrication. Following the aforementioned procedure, 2D patterned HA/TCP slurry was stacked layer by layer, and finally the viscous HA/TCP slurry was formed into the desired 3D shape.

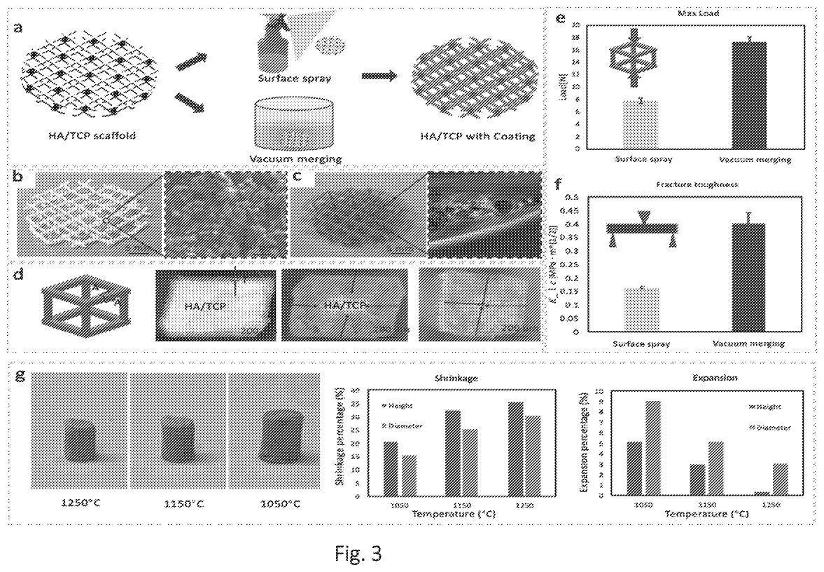

After the 3D printing process, the green part of the HA/TCP scaffold was prepared. Then the post-processing, including debinding and sintering, were conducted to remove the inner polymer and to fuse the HA/TCP particles [33]. After the debinding, HA/TCP particles were loosely arranged, and the inner porous structures were partially reduced in the sintering process, where the temperature was set much higher. In the sintering, the point contact between HA/TCP particles became a grain boundary, and the grain grew bigger with the increasing of the sintering temperature, resulting in the reduction of porosity and the shrinkage of HA/TCP scaffold. The porosity of the HA/TCP scaffold was adjusted by changing the sintering temperature and the mass concentration of HA/TCP particles. For example, the shrinkage ratio and porosity of 30% HA/TCP scaffold sintered at 1050° C., is 17.75%, and 20%, respectively, and the shrinkage of HA/TCP scaffold increased with the increase of sintering temperature (refer to FIG. 3, g). The compensation operation was conducted to get the desired shape of the HA/TCP scaffold based on the shrinkage ratio of the HA/TCP scaffold after the sintering process. Besides, the mechanical performance of the HA/TCP scaffold was improved as the result of grain growth. However, the compressive strength of the HA/TCP scaffold is only several to a dozen MPa, when it was sintered at the temperatures ranging from 1050 degrees to 1250 degrees. The 3D printed HA/TCP scaffold was too fragile to stand any further operations in the following surgeries. Hence, the poor mechanical performance of the bioceramic scaffold restricts its applications in the bio tissue regeneration. In this work, a second stage, coating process, was integrated into the fabrication of the HA/TCP scaffold to significantly improve its mechanical performance. At the same time, the biodegradability of the HA/TCP scaffold remains to be unchanged, The differences in the coating material and the penetration depth of coating material lead to widely tunable mechanical and biodegradable properties.

### EXAMPLE 2. MECHANICAL PROPERTY CHARACTERISTICS

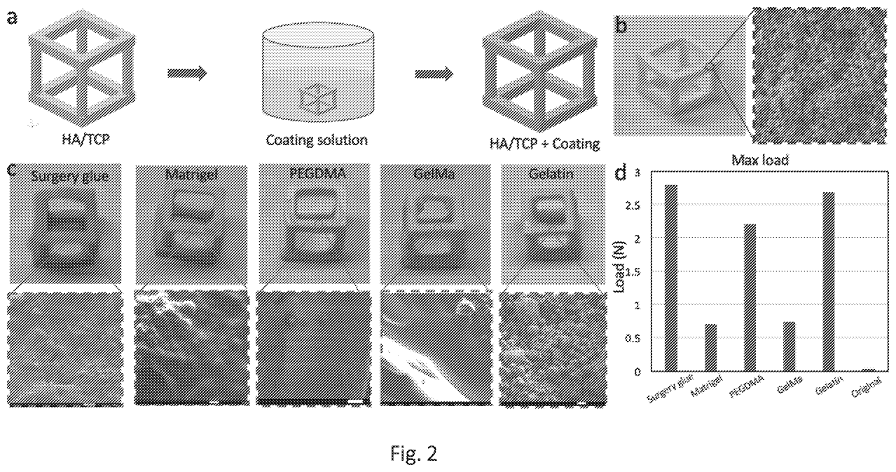

The mechanical properties of the 3D printed HA/TCP scaffolds with/without coating material were evaluated by the max load that the scaffolds can withstand, indicating the compressive strength of the 3D printed HA/TCP parts. FIG. 2 showed that the max load of the HA/TCP microcell printed part without coating material is only 0.04 N due to the poor bonding between HA/TCP particles. However, the coating material gradually filled inside the pores of the 3D printed HA/TCP parts to enhance the bonding of HA/TCP particles using the coating process. We firstly evaluated the mechanical properties of HA/TCP after coating processing by using different coating materials. After surface spray, the max loads of the HA/TCP microcell printed part increased to 2.8 N and 2.2 N for the surgical glue and PEGDMA, respectively. To be more specific, the cyanoacrylate-based polymer in the surgical glue would polymerize via an anionic mechanism, eventually creating a long chain of polymers. After the coating process the mechanical performance of the 3D printed HA/TCP part is effectively enhanced with the intense binding between polymer and HA/TCP particles. Other types of coating materials, such as acrylates based photocurable polymer and Gelatin, would react with different cross-link mechanisms contributing to enhancing the bonding of HA/TCP particles, which dominate the mechanical properties of HA/TCP printed parts (FIG. 2, c-d). The bonding evolution during the coating process was verified by scanning electron microscopy (SEM) imaging. During the coating process, the coating solution gradually penetrated inside the 3D printed HA/TCP parts with microscale pores. After the coating process, the polymer in the coating solution was crosslinked, so that the HA/TCP particles were trapped in the network of the coating material. As shown in FIG. 2, c, a thin cladding layer of the polymer was generated, and its mixing with the underneath HA/TCP particles obviously appeared, suggesting the close bonding between the coating material and the HA/TCP particles. Note that even when the penetration depth of coating material was relatively small, which was only hundreds of microns thickness, the max load of the 3D printed HA/TCP part with acrylate-based coating material already increased more than 100 times than that of the original HA/TCP printed part without coating. By exploring coating material with high mechanical properties and the increasing penetration depth of the coating material, the stronger bonding between HA/TCP particle and coating material can be obtained to further reinforce the mechanical performance of the HA/TCP scaffold. Since the coating material we selected was fast degradable materials without toxic ingredients, making it possible for the later biological applications.

Widely adjustable mechanical and biodegradable properties can be achieved by changing the penetration of the coating material. The effect of coating depth on the mechanical properties of HA/TCP printed parts was evaluated. Both surface spray and vacuum merging were applied in the coating process to accomplish various penetration depths of coating material inside the HA/TCP printed parts (refer to FIG. 3, a). The maximum load and fracture toughness grow continuously with the increasing penetration depth for both coating processes.

(1) In the surface spray, the coating thickness in a range from several microns to hundreds of microns were obtained, for example 5 μm, 10 μm, 20 μm, 30 μm, 40 μm, 50 μm, 60 μm, 70 μm, 80 μm, 90 μm, 100 μm, 150 μm, 200 μm, 250 μm, 300 μm, 400 μm, 500 μm, 600 μm, 700 μm, 800 μm, 900 μm or 1 mm, and the maximum load and fracture toughness (i.e., flexural strength) of the HA/TCP printed parts with the largest coating layer thickness by using the surface spray were 8 N and 0.16 MPa·m^{1/2} respectively (refer to FIG. 3, c-f). The mechanical property distinction of the HA/TCP printed parts with one type of coating material was mainly derived from the penetration depth of the coating material. In addition to the surface spray, the coating material can also be applied to certain places of the scaffold using the brush spray. Similar coating thickness in a range from several microns to hundreds of microns were obtained, and the mechanical performance of the coated HA/TCP printed parts is similar to the ones with the surface spray as shown in FIG. 3, e-f.

(2) In addition, the vacuum merging can markedly increase the penetration depth of the coating material in the 3D printed scaffolds. In some examples, the penetration depth is 50 μm, 100 μm, 150 μm, 200 μm, 250 μm, 300 μm, 350 μm, 400 μm, 450 μm, 500 μm, 550 μm, 600 μm, 700 μm, 800 μm, 900 μm, 1 mm, 1.25 mm, 1.5 mm, 1.75 mm, 2 mm, 2.25 mm, 2.5 mm, 2.75 mm, 3 mm, 3.25 mm, 3.5 mm, 3.75 mm, 4 mm, 4.25 mm, 4.5 mm, 4.75 mm or 5 mm, or any range between two of the preceding values. More specifically, microscale and nanoscale pores were generated inside the HA/TCP scaffold after the sintering process (FIG. 3, b), and the coating material filled in the pores of HA/TCP printed parts along with the inner air was sucked out by the vacuum generator. As shown in FIG. 3, d, the penetration depth of coating material increased with the increment of the coating time. For example, it cost the 20 s and 35 s for the coating of surgical glue with penetration depth 500 μm and 1.4 mm. After the fully coating with surgical glue, the maximum load and fracture toughness of HA/TCP printed parts increased to 19 N and 0.40 MPa·m^{1/2}, respectively (FIG. 3, c-f). The maximum loads of HA/TCP printed parts were more determined by the coating process. Compared with the pure HA/TCP printed part without the coating process, the maximum load and fracture toughness enhanced more than 450 times and 26 times, respectively.

Besides the changes in mechanical performance, the shape of HA/TCP printed parts were also changed after the sintering and coating processes. Specifically, the HA/TCP printed parts were shrunk after the sintering process because the inner gap between the HA/TCP printed parts was reduced with the growth of grain. The shrinkage ratio Rₛ of HA/TCP printed parts was enlarged with the increasing of sintering temperature. The shrinkage of HA/TCP printed parts was not homogeneous, where the shrinkage ratio of HA/TCP printed parts in the axial direction was bigger than the one in the radial direction. The shrinkage results of HA/TCP printed parts sintered at different temperatures were shown in FIG. 3, g. However, the HA/TCP printed parts expanded after the coating material filled inside the inner pores. Similarly, the expansion of HA/TCP printed parts showed anisotropic in the axial and radial directions. The expansion ratio of HA/TCP printed parts in the axial direction is larger than the one in the radial direction. The expansion ratio Rₑwas reduced with the increasing of sintering temperature since HA/TCP printed part turned more compact with smaller porous structures. Based on the experiment results, the dimensional compensation of printed model needs to be applied to the input CAD model to accurately control the shape of HA/TCP scaffolds. The dimensions of HA/TCP scaffold were adjusted according to the shape-changing rate:

(1−rₛ)(1+rₑ),

and the compensation factor ϕ can be calculated by:

$$\frac{1}{( 1 - {r}_{s} ) ( 1 + {r}_{e} )} .$$

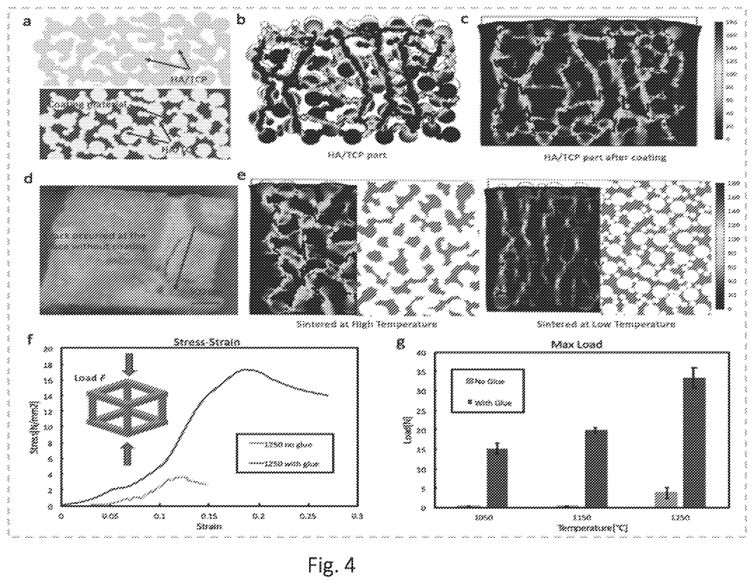

Moreover, the sintering temperature has an impact on mechanical and biodegradable properties. The compressive strengths of HA/TCP scaffold with microcell structures sintered at different temperatures were compared (refer to FIG. 4). The brittle fracture with crack first happened in the region of HA/TCP scaffold without the coating material (FIG. 4, d), and gradually lead to catastrophic failure (FIG. 4, b). However, there is no big failure, and only small fractures existed at the HA/TCP printed parts with coating material under the same load compared with the HA/TCP printed parts without coating material (FIG. 4, c). The structural simulation using COMSOL Multiphysics demonstrates that the stress is concentrated on the joint area between the HA/TCP particles (FIG. 4, b-c). The inhomogeneous stress distribution can easily break the grain boundary between the HA/TCP particles and lead to the crack, which was branched along with the distribution of HA/TCP particles without coating (FIG. 4, d).

The stress-strain behavior of the 3D HA/TCP printed parts sintered at different temperatures with coating material were studied. The grain boundary turned to be bigger with the increasing of the sintering temperature, and the simulation result showed that the stress concentrated at the grain boundary of HA/TCP particles (FIG. 4, e). The stress-strain curves of HA/TCP microcell structures sintered at 1250° C. with and without surgical glue coating are shown in FIG. 4, f. The max loads of HA/TCP scaffolds with microcell structures sintered at different temperatures, including 1050° C., 1150° C., and 1250° C. were demonstrated in FIG. 4, g. The max load of HA/TCP printed parts dropped with the decreasing of sintering temperature. For example, the max loads of HA/TCP sintered at 1050° C. and 1250° C. were 15 N and 33 N, respectively. With the increasing of sintering temperature, the grain of HA/TCP particles grows bigger, resulting in the capability to endure more stress.

In terms of the HA/TCP printed parts with fully coated material, the nucleophile initiates the reaction with Hydroxide in HA by attacking the carbon and carbon covalent bond, which breaks and forms a new bond. The strong bonding networks between HA/TCP particles and cyanoacrylate are formed after the polymerization of cyanoacrylate-based coating material. The amplified bonding of polymer and HA/TCP particles enhanced the mechanical properties. The load was further increased to break the bonds between the polymer matrix and HA/TCP particles. Therefore, the max load of the coated HA/TCP scaffold sintered at 1250° C. is bigger than the one of the coated HA/TCP scaffold sintered at 1050° C. However, the coating reinforced compressive strength improvement of HA/TCP scaffold sintered at 1250° C. is not as spectacular as the one sintered at 1050° C.

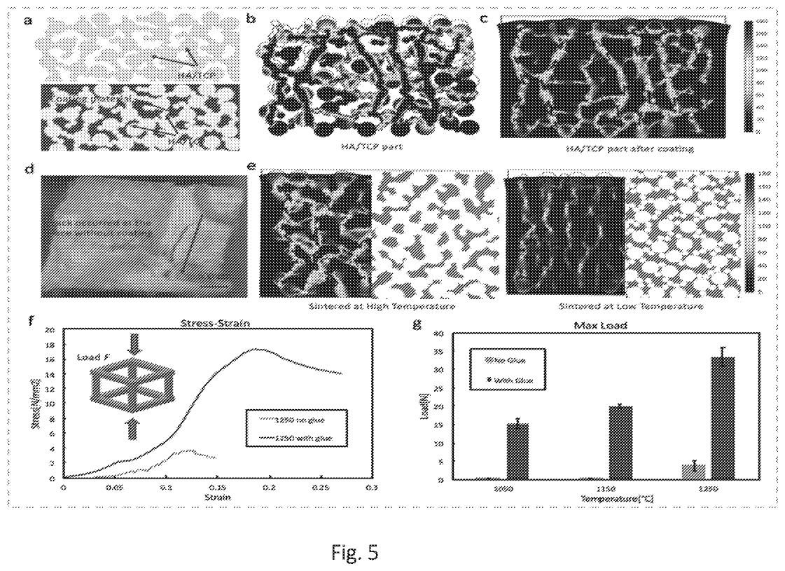

The effect of the coating reinforcement process on the flexural strength and fracture toughness was identified by performing the standard three-point bending test, The 3D printed HA/TCP parts (7.14 mm×1.07 mm×1.42 mm) with and without surgical glue coating were tested to identify the reinforcement mechanisms of the coating material (FIG. 5, a). The load-displacement curves and fracture toughness of the HA/TCP printed parts sintered at 1050° C. before and after the coating process were shown in FIGS. 5, b-c, respectively. For example, the facture toughness of HA/TCP printed part sintered at 1150° C. improved 18 times from only 0.015 MPa·m^{1/2} to 0.27 MPa·m^{1/2}. The simulation results of COMSOL Multiphysics demonstrate the damage in pure HA/TCP only locate at grain boundary, which exhibits unstable cracking characteristics; in comparison, the addition of polymer arrested the dominant crack and deflected microcracks (refer to FIG. 5, e). The results show that the pure HA/TCP ceramic printed parts generated a catastrophic failure very fast with curved cracks in a short bending distance (FIG. 5, f). After coating polymer-based material, even there were slightly cracks in the grain boundary between HA/TCP particles, the cyanoacrylate-based polymer still showed the capability of holding all the cracked parts together. As shown in FIG. 5, g, the crack deflected and occurred at the interface between HA/TCP and cyanoacrylate polymer, and the crack was bridged with macro- and micro-fibrils, leading a significant enhancement in the fracture toughness.

The sintering temperature also affects the fracture toughness and flexural strength. For instance, the flexural strength of HA/TCP printing parts sintered at 1050° C. is two times of the one sintered at 1250° C. after the coating process. Furthermore, the flexural strength of HA/TCP printed part sintered at 1050° C. increased 260 times compared with the one without coating material. The coating process significantly improved the flexural strength and fracture toughness of the 3D printed HA/TCP parts, since the coating material plays a dominant role in the bending performance of the 3D printed HA/TCP parts.

The bridging macro- and micro-polymer fibrils also contribute to improving the tensile strength of the HA/TCP printed part. As shown in FIG. 5, i, the HA/TCP printed parts sintered at 1050° C. has more displacement than the one sintered at 1250° C. due to the more coating material inside the printed parts. The Young's modulus of the HA/TCP printed parts increased a hundred times in consequence of the additives of the polymer. The pure HA/TCP revealed a brittle fracture under small tensile force, and it showed catastrophic failure with the slight load. Different from pure HA/TCP polymer, the micro-polymer fibril was obvious between the two sides of failure, forming a jagged fracture. Similarly, the young's modules difference of the HA/TCP printed parts sintered at different temperatures after the coating process is attributed to the proportion of the coating material inside the HA/TCP printed parts. The brittle fracture occurs by the formation, and rapid propagation of cracks happens in the HA/TCP printed parts sintered at 1250° C. due to the dense HA/TCP particles with less polymer coating. HA/TCP printed parts sintered at 1050° C. after the coating process showed ductility because the coating material was connected that wrapped the HA/TCP particles (refer to FIG. 5, k).

In addition to surgical glue that we extensively studied, Gelatin as the coating material shows significant mechanical performance improvement and desired surface properties with micro-holes for cell attachment (refer to FIG. 2, c-d). The aforementioned coating materials may also be diluted with certain percentage of water before the coating process. This may lead to the increased bio-degradation properties of the 3D printed HA/TCP parts.

### EXAMPLE 3. PRINTING TRIMMABLE PLATE FOR UNIVERSAL CRANIAL FACIAL BONE DEFECTS

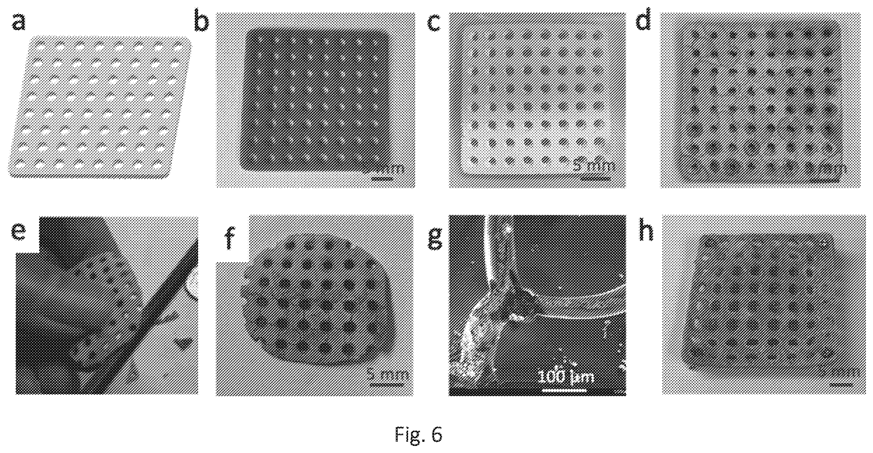

We fabricated HA/TCP trimmable plates using our proposed method for the universal purpose of bone defects. The CAD model of the HA/TCP trimmable plate is shown in FIG. 6, a. The total height of the green part of HA/TCP is 2 mm, and the total XY size of the scaffold is 40 mm×40 mm, which is decorated with an array of 2.5 mm through-holes. In order to achieve better surface quality, the slicing thickness of the scaffold place was set at 75 μm per layer, and the grayscale level of the 2D patterned light beam was modified based on the aforementioned curing performance database. However, any size scaffold could be created and any slicing thickness of the scaffold layer could be used for printing the scaffold. FIG. 6, b shows the printing result of the HA/TCP plate before debinding and sintering. The HA/TCP plates after sintering and coating are shown in FIG. 6, c-d, respectively. With the reinforcement of the coating process, the HA/TCP plate can be cut into the desired shape, which fits the demand of different bone defects. The scanning electron microscope (SEM) of the fabricated HA/TCP plate with the coating material is shown in FIG. 6, g. The through-hole array was designed to mount the screw on the fracture place, and the fixture position can be adjusted based on the real surgical case. It can be seen that the surface quality of the fabricated HA/TCP trimmable plate with reinforcement coating is satisfactory. Besides, various scaffold plates designed with different thicknesses and sizes can be fabricated using our proposed method, enabling the largescale fabrication of HA/TCP scaffold for universal bone reconstruction. In some examples, the scaffold thickness may be in the range of 100 μm to 100 mm, for example, 100 μm, 200 μm, 300 μm, 400 μm, 500 μm, 600 μm, 700 μm, 800 μm, 900 μm, 1 mm, 1.2 mm, 1.4 mm, 1.6 mm, 1.8 mm, 2 mm, 3 mm, 5 mm, 10 mm, 15 mm, 20 mm, 25 mm, 30 mm, 35 mm, 40 mm, 45 mm, 50 mm, 60 mm, 70 mm, 80 mm, 90 mm or 100 mm.

### EXAMPLE 4. 3D PRINTING SCAFFOLD FOR CRANIAL FACIAL CRITICAL DEFECT

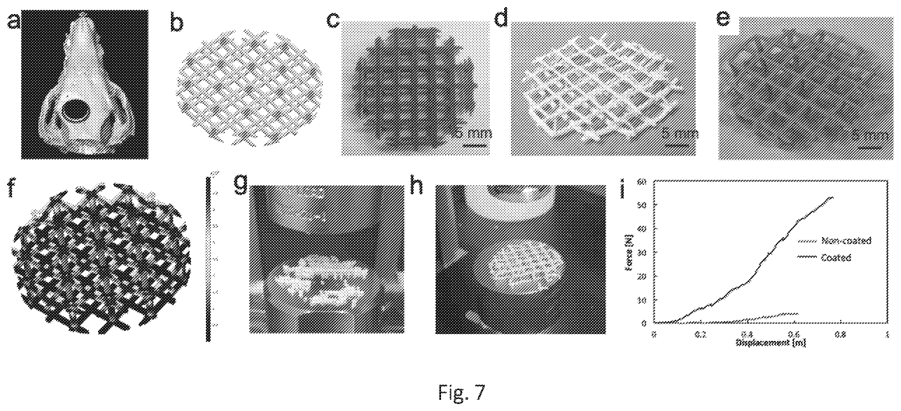

Another cranial facial test case that we tested was to build 3D HA/TCP scaffolds for the critical bone defect, as shown in FIG. 7, a. The CAD model of the HA/TCP scaffold, which is composed of microcell structures, is shown in FIG. 7, b. The height and diameter of the HA/TCP scaffold are 8 mm and 40 mm, respectively. Firstly, a green part of the HA/TCP scaffold was fabricated using the MIP-SP. Then the pure HA/TCP scaffold after debinding and sintering is shown in FIG. 7, d. The reinforcement coating process was then used to fabricate high strength HA/TCP scaffolds (refer to FIG. 7, e). The compression simulation of pure HA/TCP scaffold using COMSOL is shown in FIG. 7, t, where the joint area supports large stress. The relevant compression test of the HA/TCP scaffold with and without the reinforcement coating process was conducted (refer to FIG. 7, g (without reinforcement coating), FIG. 7, h (with reinforcement coating)). The pure HA/TCP scaffold was crushed after compression with only several Newtonian forces. However, the HA/TCP scaffold with coating material could maintain the shape after compression. Until the compression load was gradually increased to 55 N, only small fractures can be observed in the HA/TCP scaffold with coating material. The detail of force and displacement of HA/TCP scaffold with and without coating is shown in FIG. 7, i.

### EXAMPLE 5. FEMUR CRITICAL DEFECTS REGENERATION

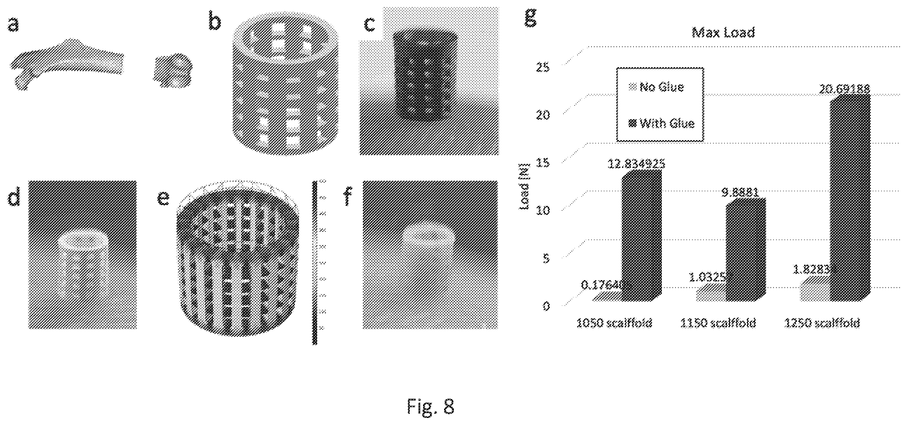

As another example, a thin-shell-shaped HA/TCP scaffolds for the critical defect model of Mouse femur can also be built (refer to FIG. 8, a). The CAD model of the HA/TCP scaffold is shown in FIG. 8, b, and the thickness and height of the scaffold are 100 μm and 2 mm, respectively. The micro-scale 150 μm holes were added on the sidewall of the scaffold for the transportation of nutrition. Firstly, the HA/TCP scaffold was fabricated by using MIP-SP (FIG. 8, c). Then the debinding and sintering were conducted to remove the inner polymer and get a pure HA/TCP scaffold as shown in FIG. 8, d. The COMSOL software system was used to simulate the stress and strain of HA/TCP under the compression (FIG. 8, e). To improve the mechanical performance, the coating material was filled inside the HA/TCP scaffold, and FIG. 8, f shows the view of the fabricated HA/TCP scaffold with coating. Furthermore, we studied the compression performance of the HA/TCP scaffold with and without coating. The maximum compression load of the HA/TCP scaffold was increased nearly ten times than the one of the original pure HA/TCP sintered at different temperatures (refer to FIG. 8, g). The mechanical performance of the scaffolds satisfied the requirement of the experiment, and there is no failure of the HA/TCP scaffold during the surgery.

### EXAMPLE 6. HA/TCP SLURRY

Microscale hydroxyapatite (HA) and tricalcium phosphate (TCP) powder were purchased from Sigma-Aldrich for the fabrication of HA/TCP scaffold. The average diameters of HA and TCP powder are 10 μm and 4 μm, respectively. The polymer binder, which will be removed after the debinding process, is necessary to form the HA/TCP powder into 3D shape during the 3D printing process. To prepare the HA/TCP based slurry, 15 wt % HA and 15 wt % TCP were firstly mixed into the photo-curable liquid resin WaxCast, which is purchased from MakerJuice Labs. The ingredient of liquid resin are the acrylate ester, photoinitiator, crosslinking agent, and stabilizer, and the photocurable polymer can be crosslinked under the exposure of visible light. Then the HA/TCP suspension was ball-milled with rotating speed at 200 rpm for 40 mins to ensure the HA/TCP powders were homogeneously distributed inside the photocurable resin. Finally, the residual gas in the HA/TCP slurry was removed by vacuuming with specific equipment for later usage.

### EXAMPLE 7. MIP-SP

A prototype machine of MIP-SP, which is composed of an optical module, material supply module, and motion module, was constructed to fabricate the green part of HA/TCP scaffold. In the optical module, a digital Micro-mirror (DMD) device (Texas Instruments) has millions of micro-mirrors, which can be controlled individually to set its state ON or OFF [38]. In the ON state, the light is reflected, making the pixel in the projection area appear bright on the top surface of the transparent disk. The brightness of each pixel in the 2D patterned light beam can be adjusted by controlling the angle of the micromirror. The fabrication area of MIP-SP is 106×60 mm², and the resolution of curing light beam is 55 μm per pixel in our prototype machine. Since the viscosity of 30 wt % HA/TCP slurry was 5000 mPa-s, this material cannot refill back to the printing area when it was driven only by air pressure and gravity. A doctor blade assisted material feeding system was applied to solve the refilling problem of HA/TCP slurry. A thin layer of HA/TCP slurry is formed by the blade that moves along the transparent glass plate, and the thickness of the material can be regulated by changing the moving speed and the gap distance between the blade and the transparent frame. To accelerate the slurry coating process, a transparent disc was mounted on the rotatory stage, and a layer of 100 μm thick was continuously coated on the transparent disc. All the HA/TCP parts studied in this work were printed using MIP-SP, and the exposure time for each layer curing was set at 10 seconds.

### EXAMPLE 8. DEBINDING AND SINTERING

The debinding process was conducted by using a tube furnace in the vacuum environment to smoothly remove the inner polymer of HA/TCP printed part and to avoid cracks, where the decomposition rate of the polymer exceeds its pyrolysis rate. The generated gas was continuously sucked out, and the inside pressure of the heating area was maintained at −0.1 MPa. Specifically, in the debinding process, the rate of heating was set to 1 degree/min, and the temperature was kept at 500° F., 900° F., 1200° F., respectively, for 2 hours. Then the temperature was cooled down to the 1000° F. for one hour to fully burn out the inner polymer. Finally, the temperature was naturally cooled down to room temperature. After the debinding process, the HA/TCP powder in the printed parts was sparsely arranged, resulting in poor mechanical performance. To solve this problem, an additional sintering process had to be conducted after the debinding process.

In the sintering process, the temperature setting was much higher so that the HA/TCP particle can grow and fuse together. The shrinkage ratio and mechanical performance of the HA/TCP scaffold can be adjusted by changing the sintering temperature. The HA/TCP scaffold was sintered in a tube furnace at the normal air condition. To achieve a large range of mechanical properties, the sintering temperatures were set at three different levels. For all the temperature settings, the temperature increasing rate was set as 5 degrees/man, and the temperature was kept at 300° C., 600° C., and 900° C. After that, the temperature was raised to 1050° C., 1150° C., and 1250° C., respectively, and was further kept at the peak value for 3 hours [33]. The sintering process is finished after natural cooling.

### EXAMPLE 9. COATING MATERIAL AND PROCESS

Dental surgical glue, internal tissue glue, and corning matrigel matrix (recon base membrane) purchased from Glustitch Inc., COHERA medical Inc., and Careforde, respectively, can be used. The ingredients of surgical glue are n-Butyl cyanoacrylate and 2-Octel cyanoacrylate.

100% (w/v) Polyethylene glycol) dimethacrylate (PEGDMA, Mw 750, Sigma-Aldrich) solution is prepared to follow the below procedures: 1% (w/v) visible light photoinitiator (Irgacure 819, BASF) was firstly fully dissolved in the phosphate-buffered saline (PBS) to induce chain polymerization by the free radicals, and 100% (w/v) Polyethylene glycol) dimethacrylate (PEGDMA., Mw 750, Sigma-Aldrich) was then mixed in the solution under a magnetic stirring.

15% (w/v) Gelatin methacryloyl (GelMA): Gelatin from porcine skin (Sigma-Aldrich) was firstly mixed at 10% (w/v) with Dulbecco's phosphate-buffered saline (DPBS; Gibco) by gently swirling mixture for 15 minutes in a 50° C. water bath and stirred until completely dissolved. A high degree of methacrylation was achieved by adding 20% (w/v) of methacrylic anhydride (MA, Sigma-Aldrich) to the synthesis reaction at a rate of 0.5 mL/min under stirred conditions at 50° C. and allowed to react for 2 hours. After that, a 5 dilution with DPBS was added to stop the reaction, and the mixture was dialyzed against distilled water using 12-14 kDa cutoff dialysis tubing for 1 week at 40° C. to remove salts and residual MA. The 0.2% gelatin solution is cooled at the room temperature, heated at ˜37-40° C., and filtered through a 0.45 μm cellular acetate membrane (CA). The filtered solution was lyophilized for 1 week to generate a white porous foam and was stored at −80° C. Then 1% (w/v) Irgacure 819 was fully dissolved in the phosphate-buffered saline (PBS) to induce chain polymerization by the free radicals, and 15% (w/v) GelMA was gradually added in the solution under a magnetic stirring until the GelMA was fully dissolved in the solution.

Gelatin solution: Boil 0.25 qt water at 100° C. and sprinkled 7 g gelatin powder (Knox) over the water. Let the mixture stand 1 min and stirred 5 mins until the gelatin powder was completely dissolved. All the above solutions were degassed in the vacuum before the coating process.

Each of the above coating polymer solutions may be used alone or in combination, as a mixture of two or more of the polymer solutions.

In the surface spraying, the normal mist sprayer was used to force the coating material through a nozzle that broke up the stream of coating material by using a one-way valve. A microscale mist was generated and further deposited on the surface of the printed HA/TCP parts. The layer thickness of deposition was mainly determined by the spray time. The coating material only covered the surface by the spraying process, and the coating was hard to penetrate the HA/TCP printed parts, resulting in the relatively constrained improvement of mechanical performance. To further enhance the mechanical properties, vacuum merging was investigated in this work. In the vacuum merging, the printed HA/TCP printed parts were merged inside the reservoir filled with the coating material, and the whole reservoir was placed into the vacuum environment. The vacuum condition and coating time were adjusted to control the penetration depth of the coating material. For instance, when the air pressure is set at 25 in·Hg, the coating speed of surgical glue is 20 μm/s for the HA/TCP printed parts sintered at 1150° C.

### EXAMPLE 10. MECHANICAL TEST

A universal testing machine (Instron 5492 Dual Column Testing Systems, Instron, MA, USA) was employed to perform a series of experiments including the compression test, three-point bending flexural test, fracture toughness test, and tensile test. Three printed parts were used to estimate the mechanical properties for each experimental group. For the compression test, a static compression model with a compression speed of 5 mm/min and a maximum compression distance of 2 mm were chosen for experiments. The 3D printed test printed part was an isolated cell from an integrated printed part with 5 mm height, 2 mm width, and 0.7 mm mesh thickness. After sintering, the green part was then put in the middle of the test platform vertically for the compression test. The strength and strain were calculated using the following equations:

$$σ = \frac{F}{{A}_{0}} = \frac{F}{4 π {r}^{2}} \qquad ( 1 )$$

$$ε = \frac{L - {L}_{0}}{{L}_{0}} = \frac{Δ L}{{L}_{0}} \qquad ( 2 )$$

where F is the load, r is the diameter, and L0 is the height of the 3D printed part.

For the three-point bending flexural test, cuboid printed parts were printed with a size of 7.14 mm length, 1.07 mm width, and 1.42 mm height. After sintering, the printed parts were used to perform a three-point bending flexural test. The specimen was placed on the printed part holder with a span length of 5 mm. The test load was applied at the mid-point of the specimen with a velocity of 5 min/min. The flexural strength was calculated by

$$σ = \frac{3 FL}{2 {bd}^{2}} \qquad ( 3 )$$

where F is force at fracture point, L is the span length, b is the printed part width, and d is the part thickness [40].

To determine the fracture toughness of the 3D printed part, we adopted the single-edge notched bend (SENB) test. The printed part is the same as the ones of the three-point bending flexural tests besides it has a notch on a single side. The specimen was placed on the 3D printed part holder with a span length of 5 mm. The test load was applied at the mid-point of the specimen which is on top of the notch. The experiment was carried out with a load velocity of 5 mm/min. The fracture toughness was calculated by using the following equation:

$${K}_{IC} = \frac{PS}{{bd}^{3 / 2}} g ( a / d ) \qquad ( 4 )$$

$$g ( a / d ) = \frac{3 {( a / d )}^{1 / 2} [ 1.99 - ( a / d ) ( 1 - a / d ) ( 2.15 - 3.93 a / d + {( a / d )}^{2} ) ]}{2 ( 1 + 2 a / d ) {( 1 - a / d )}^{3 / 2}} \qquad ( 5 )$$

where P is the maximum load during the SENB test, S is the support span, b is the printed part width, d is the specimen thickness, and a is the notch depth.

The tensile test was 3D printed samples with a 9.5 mm length, 2.5 mm width, and 1 mm thickness. The tensile tests were performed with a load rate of 5 mm/min. And the elastic modulus was obtained by calculating the slope of the linear region on the stress-strain curve. The stress and strain were calculated by using the following equations, respectively.

$$σ = \frac{F}{{A}_{0}} = \frac{F}{bd} \qquad ( 6 )$$

$$ε = \frac{L - {L}_{0}}{{L}_{0}} = \frac{Δ L}{{L}_{0}} \qquad ( 7 )$$

where F is the load, b is the width, d is the length, and L0 is the height of the 3D printed part.

### Statistical Analysis

For each statistical analysis, the test was carried out by using R statistical software. All data were expressed as mean±standard deviation (SD). The significant parameters in each experiment were determined by using the one-way analysis of variance (ANOVA), and the statistical significance was considered as p<005.

All articles, patents, patent applications, and other publications that have been cited in this disclosure are incorporated herein by reference.

In this disclosure, the indefinite article “a” and phrases “one or more” and “at least one” are synonymous and mean “at least one”.

Relational terms such as “first” and “second” and the like may be used solely to distinguish one entity or action from another, without necessarily requiring or implying any actual relationship or order between them. The terms “comprises,” “comprising,” and any other variation thereof when used in connection with a list of elements in the specification or claims are intended to indicate that the list is not exclusive and that other elements may be included. Similarly, an element preceded by an “a” or an “an” does not, without further constraints, preclude the existence of additional elements of the identical type.

The abstract is provided to help the reader quickly ascertain the nature of the technical disclosure. It is submitted with the understanding that it will not be used to interpret or limit the scope or meaning of the claims. In addition, various features in the foregoing detailed description are grouped together in various examples to streamline the disclosure. This method of disclosure should not be interpreted as requiring claimed examples to require more features than are expressly recited in each claim. Rather, as the following claims reflect, inventive subject matter lies in less than all features of a single disclosed example. Thus, the following claims are hereby incorporated into the detailed description, with each claim standing on its own as separately claimed subject matter.

### RELATED ART REFERENCES

The following publications are related art for the background of this disclosure.

Greenwald, A.S., Boden, S.D., Goldberg, V.M., Khan, Y., Laurencin, C.T. and Rosier, R.N., 2001. Bone-graft substitutes: facts, fictions, and applications. JBJS, 83(2_suppl_2), pp.S98-103.

Faour, O., Dimitriou, R., Cousins, C.A. and Giannoudis, P.V., 2011. The use of bone graft substitutes in large cancellous voids: any specific needs? Injury, 42, pp.S87-S90.

Brydone, A.S., Meek, D. and Maclaine, S., 2010. Bone grafting, orthopaedic biomaterials, and the clinical need for bone engineering. Proceedings of the Institution of Mechanical Engineers, Part H: Journal of Engineering in Medicine, 224(12), pp.1329-1343.

[symbol]

Goldberg VM. Natural history of autografts and allografts. InBone implant grafting 1992 (pp. 9-12). Springer, London.

Bohner M. Design of ceramic-based cements and putties for bone graft substitution. Eur Cell Mater. 2010 Jul 1;20(1):3-10.

Turnbull G, Clarke J, Picard F, Riches P, Jia L, Han F, Li B, Shu W. 3D bioactive composite scaffolds for bone tissue engineering. Bioactive materials. 2018 Sep 1;3(3):278-314.

Betz, R.R., 2002. Limitations of autograft and allograft: new synthetic solutions. Orthopedics, 25(5), pp.S561-S570.

Du, X., Fu, S. and Zhu. Y., 2018. 3D printing of ceramic-based scaffolds for bone tissue engineering: an overview. Journal of Materials Chemistry B, 6(27), pp.4397-4412.

Ji, K., Wang, Y., Wei, Q., Zhang, K., Jiang, A., Rao, Y. and Cai, X., 2018. Application of 3D printing technology in bone tissue engineering. Bio-Design and Manufacturing, 1(3), pp. 03-210.

Leung, Y.S., Kwok, T.H., Li, X., Yang, Y., Wang, C.C. and. Chen, Y., 2019. Challenges and Status on Design and Computation for Emerging Additive Manufacturing Technologies. Journal of Computing and Information Science in Engineering, 19(2), p.021013. DOI:10.1115/1.4041913

Yang, Y., Song, X., Li, X., Chen, Z., Zhou, C., Zhou, Q. and Chen, Y., 2018, Recent progress in biomimetic additive manufacturing technology: from materials to functional structures. Advanced Materials, 30(36), p.1706539. DOI:10.1002/adma.201706539

Guvendiren, M,, Molde, J., Soares, R.M. and Kohn, J., 2016, Designing biomaterials for 3D printing. ACS biomaterials science & engineering, 2(10), pp.1679-1693.

Chou, L., Marek, B. and Wagner, W.R., 1999. Effects of hydroxylapatite coating crystallinity on biosolubility, cell attachment efficiency and proliferation in vitro, Biomaterials, 20(10), pp.977-985.

LeGeros, R.Z., 2002. Properties of osteoconductive biomaterials: calcium phosphates. Clinical Orthopaedics and Related Research (1976-2007), 395, pp. 81-98.

Frayssinet, P., Trouillet, J.L., Rouquet, N., Azimus, E. and Autefage, A., 1993. Osseointegration of macroporous calcium phosphate ceramics having a different chemical composition. Biomaterials, 14(6), pp.423-429.

Yuan, H., Yang, Z., Li, Y., Zhang, X., De Bruijn, J.D. and De Groot, K., 1998. Osteoinduction by calcium phosphate biomaterials. Journal of materials science: materials in medicine, 9(12), pp.723-726.

Hutmacher, D.W., Schantz, J.T., Lam, C.X.F., Tan, K.C. and Lim, T.C., 2007. State of the art and future directions of scaffold-based bone engineering from a biomaterials perspective. Journal of tissue engineering and regenerative medicine, 1(4), pp.245-260.

Kokubo, T.. Kim, H.M. and Kawashita, M., 2003. Novel bioactive materials with different mechanical properties. Biomaterials, 24(13), pp.2161-2175.

Amaral, M., Lopes, M.A., Silva, R.F. and Santos, J.D., 2002. Densification route and mechanical properties of Si3N4—bioglass biocomposites. Biomaterials, 23(3), pp.857-862.

Chen, Q., Zhu, C. and Thouas, G.A., 2012. Progress and challenges in biomaterials used for bone tissue engineering: bioactive glasses and elastomeric composites. Progress in Biomaterials, 1(1), p.2.

Tarafder, S., Balla, V.K., Davies, N.M., Bandyopadhyay, A. and Bose, S., 2013. Microwave-sintered 3D printed tricalcium phosphate scaffolds for bone tissue engineering. Journal of tissue engineering and regenerative medicine, 7(8), pp.631-641.

Kang, Y., Scully, A., Young, D.A., Kim, S., Tsao, H., Sen, M. and Yang, Y., 2011. Enhanced mechanical performance and biological evaluation of a PLGA coated β-TCP composite scaffold for load-bearing applications. European polymer journal, 47(8), pp.1569-1577.

Lei, Y., Rai, B., Ho, K.H. and Teoh, S.H., 2007. In vitro degradation of novel bioactive polycaprolactone—20% tricalcium phosphate composite scaffolds for bone engineering. Materials Science and Engineering: C, 27(2), pp.293-298.

Ma, H., Feng, C., Chang, J. and Wu, C., 2018. 3D-printed bioceramic scaffolds: From bone tissue engineering to tumor therapy. Acta biomaterialia, 79, pp.37-59.

Dimitriou, R., Jones, E., McGonagle, D. and Giannoudis, P.V., 2011. Bone regeneration: current concepts and future directions. BMC medicine, 9(1), p.66.

Macchetta, A., Turner, I.G. and Bowen, C.R., 2009. Fabrication of HA/TCP scaffolds with a graded and porous structure using a camphene-based freeze-casting method. Acta biomaterialia, 5(4), pp.1319-1327.

Baradararan, S., Hamdi, M. and Metselaar, I.H., 2012. Biphasic calcium phosphate (BCP) macroporous scaffold with different ratios of HA/β-TCP by combination of gel casting and polymer sponge methods. Advances in applied ceramics, 111(7), pp367-373.

Rodrigues, L.R., Laranjeira, M.D.S., Fernandes, M.H., Monteiro, F.J. and Zavaglia, C.A.D.C., 2014, HA/TCP scaffolds obtained by sucrose crystal leaching method: Preliminary in vitro Evaluation. Materials Research, 17(4), pp.811-816.

Cihlár̆, J., Buchal, A. and Trunec, M., 1999. Kinetics of thermal decomposition of hydroxyapatite bioceramics. Journal of materials science, 34(24), pp.6121

WO2019094617 Stem cells and deviced for bone regeneration patent application:PCT/US2018/059860

Li X, Mao H, Pan Y, Chen Y. Mask Video Projection-Based Stereolithography With Continuous Resin Flow. ASME. Journal of Manufacturing Science & Engineering 2019;141(8):081007-081007-10. DOI: 10.1115/1.4043765.

Li, X., Xie, B., Jin, J., Chai, Y. and Chen, Y., 2018. 3D Printing Temporary Crown and Bridge by Temperature Controlled Mask Image Projection Stereolithography. Procedia Manufacturing, 26, pp. 1023-1033. DOI: 10.1016/j.promfg.2018.07.134

Li,X., Y,Y. Liu, L. Leung, Y., Chen, Y., Guo, Y., Chai, Y., Chen, Y., D Printing of Hydroxyapatite/Tricalcium Phosphate Scaffold with Hierarchical Porous Structure for Bone Regeneration bio design and manufacturing, Bio-Design and Fabrication (in review).

Comyn, J., 2007. Adhesion science. Royal Society of Chemistry.

Saunders, K.J., 2012. Organic polymer chemistry: an introduction to the organic chemistry of adhesives, fibres, paints, plastics and rubbers, Springer Science & Business Media.

Li, X. and Chen, Y., 2017. Micro-scale feature fabrication using immersed surface accumulation. ASME. Journal of Manufacturing Processes, 28, pp.531-540. DOI: 10.1016/j.jmapro.2017.04.022

Yang. Y., Li, X., Zheng, X., Chen, Z., Zhou, Q. and Chen, Y., 2018, 3D-Printed Biomimetic Super-Hydrophobic Structure for Microdroplet Manipulation and Oil/Water Separation. Advanced materials. 30(9), p.1704912.

Yang. Y., Li, X., Chu, M., Sun, H., Jin, J., Yu, K., Wang, Q., Zhou, Q. and Chen, Y., 2019. Electrically assisted 3D printing of nacre-inspired structures with self-sensing capability. Science advances, 5(4), p.eaau9490.

Zhang, J., Yang, Y., Zhu, B., Li, X., Jin, J., Chen, Z., Chen, Y. and Zhou, Q., 2018. Multifocal point beam forming by a single ultrasonic transducer with 3D printed holograms. Applied Physics Letters, 113(24), p.243502.

Boresi, A.P., Schmidt, R.J. and Sidebottom, O.M., 1985. Advanced mechanics of materials (Vol. 6). New York et al.: Wiley.

Dixon, W.J. and Massey Jr, F.J., 1951. Introduction to statistical analysis.

1-72. (canceled)

73. A ceramic scaffold, comprising:
a framework comprising hydroxyapatite (HA), tricalcium phosphate (TCP), or a mixture thereof; and
a coating comprising a coating polymer;
wherein:
the coating is formed on at least one surface of the framework;
the coating at least partially covers the at least one surface of the framework; and
the coating polymer comprises a polymer formed by using a surgical glue, poly(ethylene glycol) dimethacrylate (PEGDMA), a gelatin, or a mixture thereof; and
wherein:
mechanical strength of the ceramic scaffold is in a range of 15 N to 33 N when measured as a maximum load of the stress vs. strain curve; and/or
flexural strength of the ceramic scaffold is in a range of 10 MPa to 50 MPa when the flexural strength is measured by a standard three-point bending test; and/or
mechanical strength of the ceramic scaffold is at least 5 times higher than that of the framework, or at least 10 times higher than that of the framework, or 10 times to 20 times higher than that of the framework when the mechanical strength of the ceramic scaffold is measured as a maximum load of the stress vs. strain curve.

74. The ceramic scaffold of claim 73, wherein thickness of the coating polymer is in a range of 1 micrometer to 1,000 micrometers.

75. The ceramic scaffold of claim 73, wherein thickness of the coating polymer is in a range of 5 micrometers to 500 micrometers.

76. The ceramic scaffold of claim 73, wherein the coating polymer comprises a surgical glue; and wherein the surgical glue comprises an acrylate polymer.

77. The ceramic scaffold of claim 73, wherein the coating polymer comprises a surgical glue; and wherein the surgical glue comprises a cyanoacrylate polymer.

78. The ceramic scaffold of claim 73, wherein the coating polymer comprises a surgical glue; and wherein the surgical glue comprises a polymer of an n-butyl cyanoacrylate monomer, a 2-octyl cyanoacrylate monomer, or a mixture thereof.

79. The ceramic scaffold of claim 73, wherein the coating polymer comprises a gelatin.

80. The ceramic scaffold of claim 73, wherein the coating substantially covers the at least one surface of the framework.

81. The ceramic scaffold of claim 73, wherein the framework comprises stacked layers of hydroxyapatite (HA), tricalcium phosphate (TCP), or a mixture thereof, each layer having a thickness in a range of 10 μm to 200 μm.

82. A ceramic scaffold, comprising:
a framework comprising hydroxyapatite (HA), tricalcium phosphate (TCP), or a mixture thereof; and
a coating comprising a coating polymer;
wherein:
the coating is formed on at least one surface of the framework;
the coating at least partially covers the at least one surface of the framework;
the coating polymer comprises a polymer formed by using a surgical glue, poly(ethylene glycol) dimethacrylate (PEGDMA), a gelatin, or a mixture thereof; and
the framework comprises stacked layers of hydroxyapatite (HA), tricalcium phosphate (TCP), or a mixture thereof, each layer having a thickness in a range of 10 μm to 200 μm.

83. The ceramic scaffold of claim 82, wherein thickness of the coating polymer is in a range of 1 micrometer to 1,000 micrometers.

84. The ceramic scaffold of claim 82, wherein thickness of the coating polymer is in a range of 5 micrometers to 500 micrometers.

85. The ceramic scaffold of claim 82, wherein the coating polymer comprises a surgical glue; and wherein the surgical glue comprises an acrylate polymer.

86. The ceramic scaffold of claim 82, wherein the coating polymer comprises a surgical glue; and wherein the surgical glue comprises a cyanoacrylate polymer.

87. The ceramic scaffold of claim 82, wherein the coating polymer comprises a surgical glue; and wherein the surgical glue comprises a polymer of an n-butyl cyanoacrylate monomer, a 2-octyl cyanoacrylate monomer, or a mixture thereof.

88. The ceramic scaffold of claim 82, wherein the coating polymer comprises a gelatin.

89. The ceramic scaffold of claim 82, wherein the coating substantially covers the at least one surface of the framework.

90. A ceramic scaffold, comprising:
a framework comprising hydroxyapatite (HA), tricalcium phosphate (TCP), or a mixture thereof; and
a coating comprising a coating polymer;
wherein:
the coating is formed on at least one surface of the framework;
the coating at least partially covers the at least one surface of the framework; and
the coating polymer comprises a polymer formed by using a gelatinous protein mixture, gelatin methacrylate (GelMA), or a mixture thereof.

91. The ceramic scaffold of claim 90, wherein thickness of the coating polymer is in a range of 1 micrometer to 1,000 micrometers.

92. The ceramic scaffold of claim 90, wherein the framework comprises stacked layers of the hydroxyapatite (HA), tricalcium phosphate (TCP), or a mixture thereof, each layer having a thickness in a range of 10 μm to 200 μm.
